(19) United States
(12) Patent Application Publication
(10) Pub. No.: US 2015/0039039 A1
(43) Pub. Date: Feb. 5, 2015

(54) Compact Controller Device for Defibrillator

(71) Applicant: ZOLL Medical Corporation, Chelmsford, MA (US)

(72) Inventors: John Macho, Pittsburgh, PA (US); Phillip Amsler, Oakmont, PA (US); Shane Volpe, Saltsburg, PA (US)

(21) Appl. No.: 14/448,857

(22) Filed: Jul. 31, 2014

Related U.S. Application Data
(60) Provisional application No. 61/861,110, filed on Aug. 1, 2013.

(51) Int. Cl. A61N 1/39

(52) U.S. Cl. CPC A61N 1/3968
USPC 607/5; 29/832

(57) ABSTRACT

A defibrillator includes: a housing; a discharge module disposed on a first portion of a printed circuit board and positioned within the housing; an energy storage module disposed on a second portion of the printed circuit board; and a controller module disposed on a third portion of the printed circuit board. The energy storage module is operatively connected to the discharge module by a first flexible member. The controller module is operatively connected to the energy storage module by a second flexible member. The first flexible member is folded such that the first portion of the printed circuit board is positioned substantially parallel to the second portion of the printed circuit board and the second flexible member is folded such that the third portion of the printed circuit board is positioned substantially perpendicular to the first and second portions of the printed circuit board.

### CROSS REFERENCE TO RELATED APPLICATIONS

This application claims the benefit of U.S. Provisional Patent Application Ser. No. 61/861,110 entitled “Compact Controller Device for Defibrillator” filed Aug. 1, 2013, the contents of which are incorporated herein by reference.

### BACKGROUND OF THE INVENTION

1. Field of the Invention

The present disclosure generally relates to the treatment of heart defects by the administration of electrical therapy and, more particularly, to a defibrillator for imparting the electrical therapy to the heart.

2. Description of Related Art

Technology is available for correcting excessively slow heart rates (bradycardia) using implantable devices, commonly referred to as pacemakers, which deliver microjoule electrical pulses to a slowly beating heart in order to speed the heart rate up to an acceptable level. Also, it is well known to deliver high energy shocks (e.g., 180 to 360 joules) via external paddles applied to the chest wall in order to correct excessively fast heart rates, and prevent the possible fatal outcome of ventricular fibrillation or certain ventricular tachycardias. Bradycardia, ventricular fibrillation, and ventricular tachycardia are all electrical malfunctions (arrhythmias) of the heart. Each may lead to death within minutes unless corrected by the appropriate electrical stimulation.

One of the most deadly forms of heart arrythmias is ventricular fibrillation, which occurs when the normal, regular electrical impulses are replaced by irregular and rapid impulses, causing the heart muscle to stop normal contractions and to begin to quiver. Normal blood flow ceases, and organ damage or death may result in minutes if normal heart contractions are not restored. Although frequently not noticeable to the victim, ventricular fibrillation is often preceded by ventricular tachycardia, which is a regular but fast rhythm of the heart. Because the victim has no noticeable warning of the impending fibrillation, death often occurs before the necessary medical assistance can arrive.

Because time delays in applying the corrective electrical treatment may result in death, implantable pacemakers and defibrillators have significantly improved the ability to treat these otherwise life-threatening conditions. Being implanted within the patient, the device continuously monitors the patient's heart for treatable arrhythmias and, when such is detected, the device applies corrective electrical pulses directly to the heart.

Normal heart function often can be restored to a person suffering ventricular fibrillation or ventricular tachycardia by a procedure known as cardioversion, the synchronized application of electrical therapy to the heart muscle. Pacemakers and defibrillators that apply corrective electrical pulses externally to the patient's chest wall also are used to correct such life-threatening arrhythmias, but suffer from a drawback insofar as it may not be possible to apply the device in time during an acute arrhythmic emergency to save the patient's life. Such treatment is needed within a few minutes to be effective.

Consequently, when a patient is deemed at high risk of death from such arrhythmias, electrical devices often are implanted so as to be readily available when treatment is needed. However, patients that have recently had a heart attack or are awaiting such an implantable device may be kept in a hospital where corrective electrical therapy is generally close at hand. Long-term hospitalization is frequently impractical due to its high cost, or due to the need for patients to engage in normal daily activities.

Defibrillators have been developed for patients that have recently experienced cardiac arrest, that are susceptible to heart arrhythmias and are at temporary risk of sudden death, and that are awaiting an implantable device. However, current wearable defibrillators may lack the required size and durability to provide maximum comfort and usability to the patient.

Accordingly, a need exists for a portable, wearable defibrillator having the components that are used to deliver the functionality of the defibrillator fit into a small footprint such that the defibrillator is small and light enough to be worn by ambulatory patients. In addition, a need exists for a wearable defibrillator that is extremely durable so that it continues to function properly even if dropped or otherwise subjected to an impact by the patient.

### SUMMARY OF THE INVENTION

Provided is a defibrillator that comprises: a housing; and a printed circuit board comprising a plurality of portions operatively connected by flexible members. The plurality of portions further comprise: a first portion of the printed circuit board upon which may be disposed a discharge module for selectively delivering an energy pulse to a patient; a second portion of the printed circuit board upon which may be disposed an energy storage module; and a third portion of the printed circuit board upon which may be disposed a controller module for controlling delivery of the energy pulse to the patient. The flexible members are folded such that at least one of the plurality of portions of the printed circuit board is positioned substantially perpendicular to at least one of the others of the plurality of portions of the printed circuit board, thereby allowing the discharge module, energy storage module, and controller module to be positioned within the housing.

The flexible members may comprise at least a first flexible member connecting the first portion of the printed circuit board to the second portion of the printed circuit board. The first flexible member may be folded such that the first portion of the printed circuit board is positioned substantially parallel to the second portion of the printed circuit board. In addition, the flexible members may comprise at least a second flexible member connecting the second portion of the printed circuit board to the third portion of the printed circuit board. The second flexible member may be folded such that the third portion of the printed circuit board is positioned substantially perpendicular to the first and second portions of the printed circuit board.

The plurality of portions may further comprise a fourth portion of the printed circuit board upon which may be disposed a communication module. The flexible members may comprise at least a third flexible member connecting the fourth portion of the printed circuit board to the third portion of the printed circuit board. The third flexible member may be folded such that the fourth portion of the printed circuit board is positioned substantially parallel to the third portion of the printed circuit board and substantially perpendicular to the first and second portions of the printed circuit board, thereby allowing the communication module to be positioned within the housing. The communication module may include at least one of a GPS transceiver, a Bluetooth™ transceiver, a Wi-Fi transceiver, and a cellular transceiver. At least one of a metallic portion of the third portion of the printed circuit board and a metallic portion of a display of the defibrillator may function as part of a cellular antenna for a cellular transceiver.

The controller module may comprise a memory and a microprocessor. The memory and microprocessor may be operatively connected to a separate printed circuit board that is operatively connected to the third portion of the printed circuit board. The memory and microprocessor may be operatively connected to the separate printed circuit board by ball grid arrays that are under-filled with an epoxy material.

The defibrillator may further comprise a display positioned on an exterior surface of the housing for providing a user interface, a speaker mounted within an air-filled reverberator positioned at a corner of the housing, a port for connecting the defibrillator to a treatment device, and a power supply configured to be positioned within a dock provided on the housing and operatively connected to a member provided on the energy storage module.

Also provided is a defibrillator that includes: a housing; at least one high-voltage module; and at least one low-voltage module operatively connected to the at least one high-voltage module by at least one flexible member. The at least one flexible member is folded such that the at least one high-voltage module and the at least one low-voltage module are positioned within the housing and such that spacing between the at least one high-voltage module and the at least one low-voltage module provides at least one of isolation of high-voltage from low voltage and minimizes interference between the at least one high-voltage module and the at least one low-voltage module.

The at least one high-voltage module may include at least one of a discharge module for selectively delivering an energy pulse to a patient and an energy storage module for storing an energy pulse to be delivered to the patient. The at least one low-voltage module may include at least one of a controller module for controlling delivery of an energy pulse to a patient and a communication module for allowing the defibrillator to communicate with an external device. The controller module may be positioned within the housing at a location such that stresses caused by external forces on the housing are reduced.

In addition, provided is a method of manufacturing a defibrillator. The method comprises providing a distributed circuit board. The distributed circuit board comprises: a discharge module disposed on a first portion of a printed circuit board for selectively delivering an energy pulse to a patient; an energy storage module disposed on a second portion of the printed circuit board that is operatively connected to the discharge module by a first flexible member; and a controller module for controlling delivery of the energy pulse to the patient disposed on a third portion of the printed circuit board that is operatively connected to the energy storage module by a second flexible member. The method also comprises: folding the first flexible member such that the first portion of the printed circuit board is positioned substantially parallel to the second portion of the printed circuit board; folding the second flexible member such that the third portion of the printed circuit board is positioned substantially perpendicular to the first and second portions of the printed circuit board, thereby providing a folded distributed circuit board; providing a housing having an upper housing portion and a lower housing portion; positioning the folded printed circuit board within one of the upper housing portion and the lower housing portion; and securing the upper housing portion to the lower housing portion.

The distributed circuit board may further comprise a communication module disposed on a fourth portion of the printed circuit board and operatively connected to the controller module by a third flexible member. The method may further comprise folding the third flexible member such that the fourth portion of the printed circuit board is positioned substantially parallel to the third portion of the printed circuit board and substantially perpendicular to the first and second portions of the printed circuit board.

Still further provided is a defibrillator comprising: a housing; a plurality of operatively connected modules; and a printed circuit board comprising a plurality of portions operatively connected by flexible members. The plurality of portions further comprise: a first portion of the printed circuit board upon which may be disposed at least one of a discharge module for selectively delivering an energy pulse to a patient-and an energy storage module; and a second portion of a printed circuit board upon which may be disposed at least one of a controller module for controlling delivery of the energy pulse to the patient and a communication module for allowing the defibrillator to communicate with an external device. At least one of the flexible members are folded such that the first portion is positioned substantially perpendicular to the second portion, thereby allowing the plurality of operatively connected modules to be positioned within the housing.

In addition, provided is a defibrillator comprising: a housing; a plurality of operatively connected modules comprising a discharge module for selectively delivering an energy pulse to a patient, an energy storage module, and a controller module for controlling delivery of the energy pulse; and a printed circuit board comprising a plurality of portions operatively connected by flexible members. The plurality of portions further comprise: a first portion of a printed circuit board upon which may be disposed at least one of the plurality of modules; and a second portion of a printed circuit board upon which may be disposed at least one of a remainder of the plurality of modules. At least one of the flexible members are folded such that the first portion is positioned substantially perpendicular to the second portion, thereby allowing the plurality of operatively connected modules to be positioned within the housing.

The at least one of the plurality of modules disposed on the first portion may comprise at least one of the discharge module and the energy storage module. The at least one of the remainder of the plurality of modules disposed on the second portion may comprise the controller module. Alternatively, At least one of the plurality of modules disposed on the second portion may comprise at least one of the controller module and a communication module for allowing the defibrillator to communicate with an external device.

These and other features and characteristics of the present invention, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and the claims, the singular form of “a”, “an”, and “the” include plural referents unless the context clearly dictates otherwise.

### BRIEF DESCRIPTION OF THE DRAWINGS

FIG. 1 is a front perspective view of the external housing of a defibrillator in accordance with the present invention;

FIG. 2 is a rear perspective view of the external housing of FIG. 1;

FIG. 3 is a rear perspective view of the external housing of FIG. 1 with a top cover plate and power supply removed;

FIG. 4 is a top plan view of a distributed circuit board configured to be positioned within the housing of the defibrillator in accordance with the claimed invention;

FIG. 5 is a bottom plan view of the distributed circuit board of FIG. 4;

FIG. 6 is a front perspective view of the distributed circuit board of FIG. 4 in a folded configuration;

FIG. 7 is a rear perspective view of the distributed circuit board of FIG. 6;

FIG. 8 is a front perspective view of the distributed circuit board of FIG. 6 with all of the electronic components removed from the portions of the circuit boards thereof;

FIG. 9 is a rear perspective view of the distributed circuit board of FIG. 8;

FIG. 9a is an end view of the distributed circuit board of FIG. 9;

FIG. 10 is a front perspective view of the distributed circuit board of FIG. 6 positioned within a front cover plate of the external housing of FIG. 1; and

FIG. 11 is a front plan view of the distributed circuit board and housing of FIG. 10.

### DESCRIPTION OF THE INVENTION

As used herein, spatial or directional terms, such as “inner”, “left”, “right”, “up”, “down”, “horizontal”, “vertical” and the like, relate to the invention as it is described herein. However, it is to be understood that the invention can assume various alternative orientations and, accordingly, such terms are not to be considered as limiting. For the purposes of this specification, unless otherwise indicated, all numbers expressing quantities of ingredients, reaction conditions, dimensions, physical characteristics, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

Also, it should be understood that any numerical range recited herein is intended to include all sub-ranges subsumed therein. For example, a range of “1 to 10” is intended to include any and all sub-ranges between and including the recited minimum value of 1 and the recited maximum value of 10, that is, all subranges beginning with a minimum value equal to or greater than 1 and ending with a maximum value equal to or less than 10, and all subranges in between, e.g., 1 to 6.3, or 5.5 to 10, or 2.7 to 6.1.

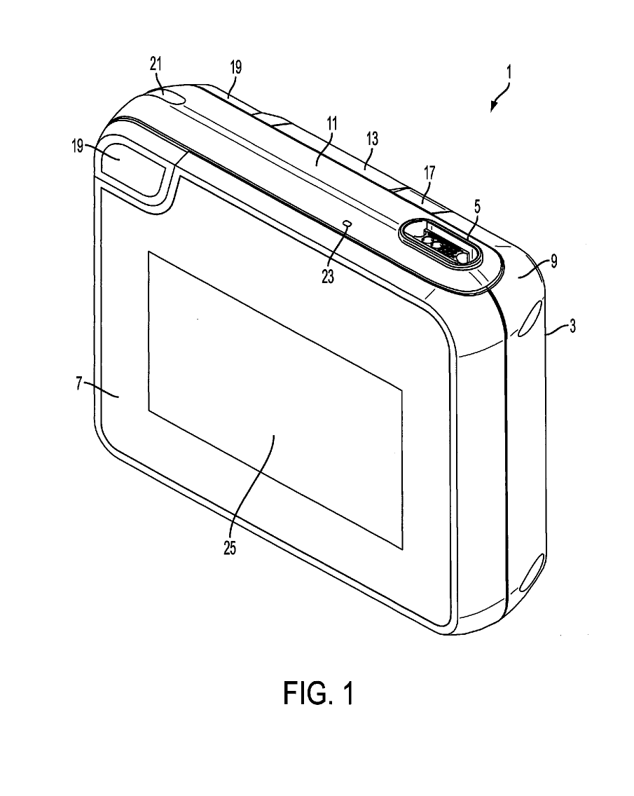

With reference to FIGS. 1-3, a defibrillator, generally denoted as reference numeral 1, is provided that is configured to implement the critical functions of monitoring an ambulatory patient's ECG information and, when needed, administering a therapeutic shock to the patient. For example, defibrillators, such as the LifeVest® wearable defibrillator available from ZOLL® Corporation, are typically worn nearly continuously for two to three months at a time. During the period of time in which they are worn by the patient, the defibrillator needs to continuously monitor the vital signs of the patient, to be user-friendly and accessible, to be as light-weight, comfortable, and portable as possible, and to be capable of delivering one or more life-saving therapeutic shocks when needed.

Referring now to FIGS. 1-4, the defibrillator 1 comprises a distributed printed circuit board 41 positioned within an external housing 3 configured to be worn by a patient and connected to a therapeutic or treatment device, such as an upper body harness or vest that includes ECG electrodes and therapy pads (not shown). The ECG electrodes and therapy pads of the harness or vest are operatively coupled to the distributed printed circuit board 41 within the external housing 3 via a port 5. Such wearable therapeutic devices are described in U.S. Pat. No. 5,741,306 and United States Patent Publication No. 2012/0011382, which are assigned to the assignee of the present application and are hereby incorporated by reference in their entirety.

In some embodiments, the external housing 3 of the defibrillator 1 comprises a front cover 7, a rear cover 9, and a top cover 11. A rechargeable and removable battery 13 is positioned within a slot 15 provided in the rear cover 9. The battery 13 is secured to the rear cover 9 by a battery latch 17. The battery latch 17 is positioned at the top left corner of the battery 13 to allow for the battery 13 to be removed from the external housing 3 with one rocking motion. This rocking motion increases usability for patients with decreased dexterity, such as a patient with arthritis. The battery 13 has sufficient capacity to administer one or more therapeutic shocks to the therapeutic electrodes as well as provide power to all of the internal components of the defibrillator 1. As mentioned hereinabove, the external housing 3 of the defibrillator 1 is configured to be worn by the patient and is accordingly sized such that it does not interfere with the patient's movement and activity. More particularly, the external housing 3 may have a length of about 5 to 6 inches, a height of about 4 to 5 inches, and a width of about 1 to 2 inches.

In some embodiments, the external housing 3 further comprises a pair of patient response buttons 19 positioned, for example, in the top left corner of the housing 3. The response buttons 19 are positioned a small distance apart, desirably less than 1.5 inches. The location of the response buttons 19 and the distance between the response buttons 19 was chosen to enable patients with limited dexterity to easily and quickly operate the response buttons 19.

In some embodiments, the defibrillator 1 further comprises an audio system having a speaker port 21 and a microphone port 23 positioned on the external housing 3. The speaker port 21 is desirably positioned at least 2.5 inches away from the microphone port 23 to minimize feedback. In addition, the speaker port 21 and the microphone port 23 can be located on the top cover 11 of the external housing 3 in order to face the patient for better orientation and functionality. The speaker port 21 is also positioned on an upper corner of the external housing 3 and wraps from the top of the external housing 3 to a side thereof. This allows the speaker port 21 to be more difficult to block if the top of the defibrillator 1 is obstructed. In addition, the speaker is mounted in a reverberator which uses a specific volume of air to artificially amplify audio at specific frequencies. The outlet of the reverberator is the speaker port 21. In one non-limiting embodiment, the reverberator is tuned to amplify the alarm frequencies at 2.272 kHz and 2.5 kHz in order to get to 95 dB at 1 m for the alarm. The microphone port 23 and the speaker port 21 are covered by a mesh or other suitable covering to prevent the ingress of fluid and/or particles into the external housing 3.

The external housing 3 of the defibrillator 1 also includes a display screen 25 for providing information to a patient and for providing a user input device to the patient. The display screen 25 provides information such as, but not limited to, time, battery life, volume, signal strength, device status, and any other useful information to the patient. In addition, the display screen 25 also allows the user to access various data regarding the defibrillator 1 such as, but not limited to, the settings of the device, data stored by the device, and various other data accumulated by the defibrillator 1. The display screen 25 further acts as a communication interface to allow the patient to send and receive data.

The display screen 25 may be any suitable capacitive touch screen device. For instance, the display screen 25 may include a 1.1 mm thick Dragontrail™ lens, manufactured by Asahi Glass Co. of Tokyo, JP, which supports a projected capacitive touch screen having a 4.3 inch LCD on the reverse side. A glass display may be provided to cover the entire front of the defibrillator 1, except for the response buttons 19, to provide the defibrillator 1 with a smooth, finished look and feel.

In operation and, as will be discussed in greater detail hereinafter, if the defibrillator 1 detects an abnormal condition, the defibrillator 1 is configured to stimulate the patient for a predetermined time period. The stimulus may be any stimulus perceptible by the patient. Examples of stimuli that the defibrillator 1 may produce include visual (via the display screen 25), audio (via the speaker port 21), tactile stimulation (via a vibrator (not shown) device included in the therapeutic device) or a mild stimulating alarm shock (via the therapeutic device). The response buttons 19 are provided to allow a user to turn off the stimulus by pressing both of the response buttons 19 within the predetermined time period. By pressing both of the response buttons 19, the stimulus is ceased and no further action is taken by the defibrillator 1. If the patient does not press both of the response buttons 19 within the predetermined time period, the defibrillator 1 administers one or more therapeutic shocks to the therapeutic electrodes of the therapeutic device.

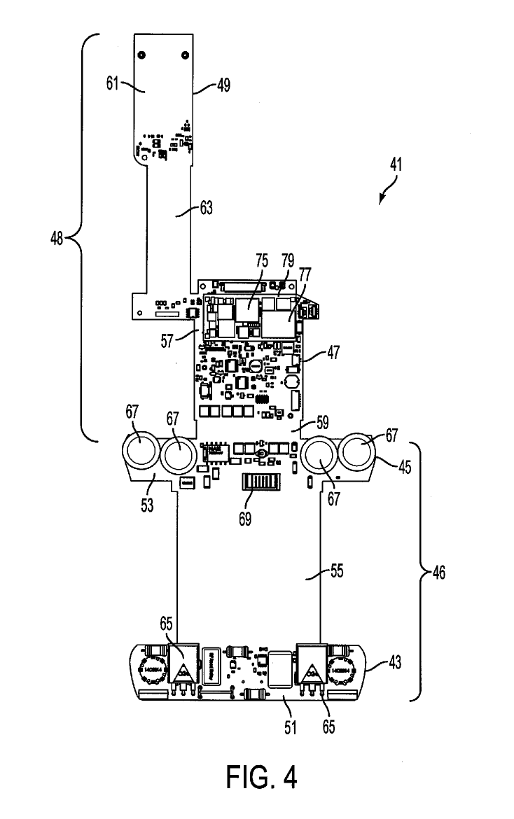

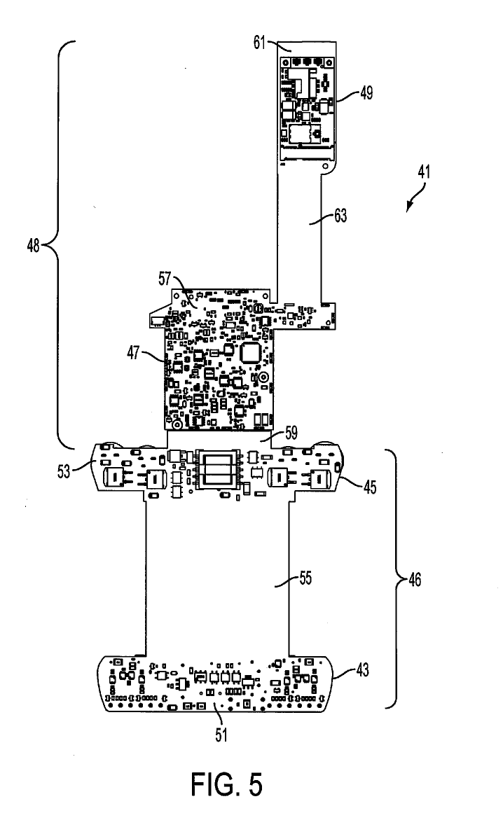

With reference to FIGS. 4-7 and with continuing reference to FIGS. 1-3, the functional components of the defibrillator 1 are illustrated. The functional components of the defibrillator 1 are provided on a distributed printed circuit board, denoted generally as reference numeral 41, such as a rigid flex printed circuit board. Rigid flex printed circuit boards are boards using a combination of flexible and rigid board technologies. The rigid flex board can comprise multiple layers of flexible circuit substrates embedded within one or more rigid boards. Rigid flex printed circuit boards are designed in a three-dimensional space, which offers greater spatial efficiency. In addition, through the use of rigid flex printed circuit boards, all board-to-board connections have been eliminated, thereby increasing the durability of the defibrillator 1.

The distributed printed circuit board 41 comprises a discharge module 43, an energy storage module 45, a controller module 47, and, optionally, a communication module 49. The discharge module 43 is disposed on a first portion 51 of the distributed printed circuit board 41 and is for selectively delivering an energy pulse to the patient. The energy storage module 45 is disposed on a second portion 53 of the distributed printed circuit board 41. The energy storage module 45 is operatively connected to the discharge module 43 by a first flexible member 55. The controller module 47 is provided to control the delivery of the energy pulse to the patient and is disposed on a third portion 57 of the distributed printed circuit board 41. The controller module 47 is operatively connected to the energy storage module 45 by a second flexible member 59. The communication module 49 can be disposed on a fourth portion 61 of the distributed printed circuit board 41 and can be operatively connected to the controller module 47 by a third flexible member 63.

The discharge module 43 and the energy storage module 45 can be considered high-voltage modules as each of these modules requires a high voltage for operation. These modules 43, 45 are provided on a high-voltage portion 46 of the distributed printed circuit board 41. The controller module 47 and the communication module 49 can be considered low-voltage modules as each of these modules requires a low voltage for operation. These modules 47, 49 are provided on a low-voltage portion 48 of the distributed printed circuit board 41. The flexible members 55, 59, and 63 (or connectors) are positioned such that, when the distributed printed circuit board 41 is folded to be positioned within the external housing 3, as discussed in greater detail hereinafter, the spacing between the high-voltage modules and the low-voltage modules provides at least one of isolation of high-voltage from low-voltage or minimizes interference, such as electromagnetic interference, between the modules. The spacing provided between the high-voltage modules and the low-voltage modules is desirably at least 0.350 inches. In some embodiments, one or more of the members 55, 59, and 63 can include a flexible portion of the distributed printed circuit board 41. In some embodiments, one or more of the members 55, 59, and 63 can be a separate connector, for example, a wire, cable, flex connector such as a ZIF (zero insertion force) connector, or any suitable electrical connector known to those skilled in the art.

The first portion 51 of the distributed printed circuit board 41 that encompasses the discharge module 43 may be a long, narrow printed circuit board. It has a length in the range of about 4 to 6 inches and a width in the range of about 0.5 to 1.5 inches. This configuration of the first portion 51 allows it to be fit securely within a bottom portion of the external housing 3 substantially perpendicular to the front cover 7 and the rear cover 9. The first portion 51 of the distributed printed circuit board 41 comprises a plurality of high voltage switches 65, such as Insulated Gate Bipolar Transistors (IGBTs), Field Effect Transistors (FETs), transistors, or Metal-Oxide Semiconductor Field-Effect Transistors (MOSFETs). Desirably, IGBTs are used as the high voltage switch. The discharge module 43 is configured to selectively deliver an energy pulse stored in the energy storage module 45 to the patient based on a signal from the controller module 47. The energy pulse is sent from the discharge module 43 through the port 5 to the therapy electrodes of the therapeutic device.

The second portion 53 of the distributed printed circuit board 41 that encompasses the energy storage module 45 is also a long, narrow printed circuit board. It has a length in the range of about 5 to 6 inches and a width in the range of about 0.5 to 1.5 inches. This configuration of the second portion 53 allows it to be fit securely within a bottom portion of the external housing 3 substantially perpendicular to the front cover 7 and the rear cover 9 and substantially parallel to the first portion 51. The second portion 53 of the distributed printed circuit board 41 includes a capacitive device mounted thereon, such as a bank of capacitors 67. Each of the capacitors in the bank of capacitors 67 may have a capacitance value of greater than 300 microfarads, such as 650 microfarads.

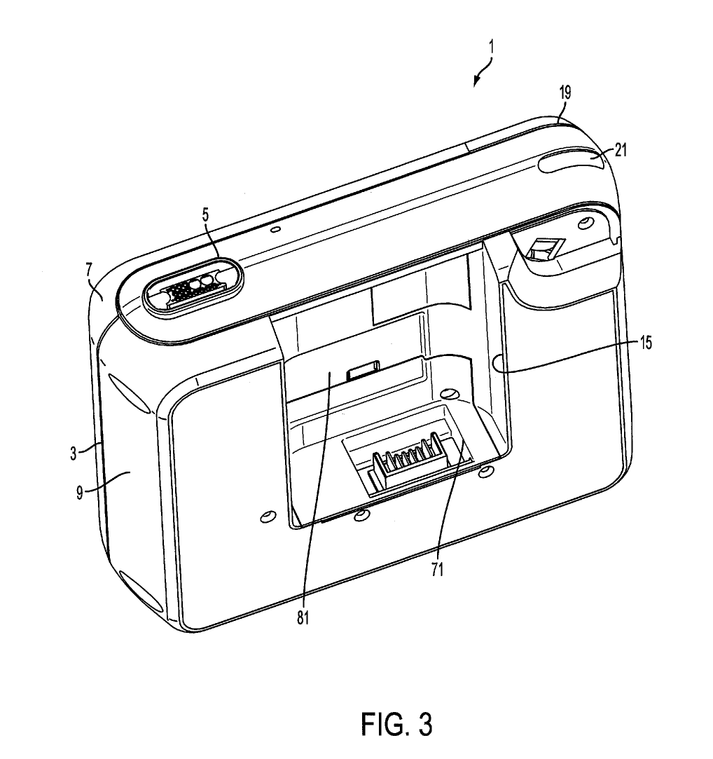

The second portion 53 further comprises a battery connector 69 mounted thereon. The battery connector 69 is configured to extend through an opening 71 (see FIG. 3) provided within the slot 15 of the external housing 3. The battery connector 69 is protected from the ingress of fluids by using an epoxy coating to seal off the underside of its blades. This allows the defibrillator 1 to be resistant to water or ingress of other materials into the interior of the external housing 3.

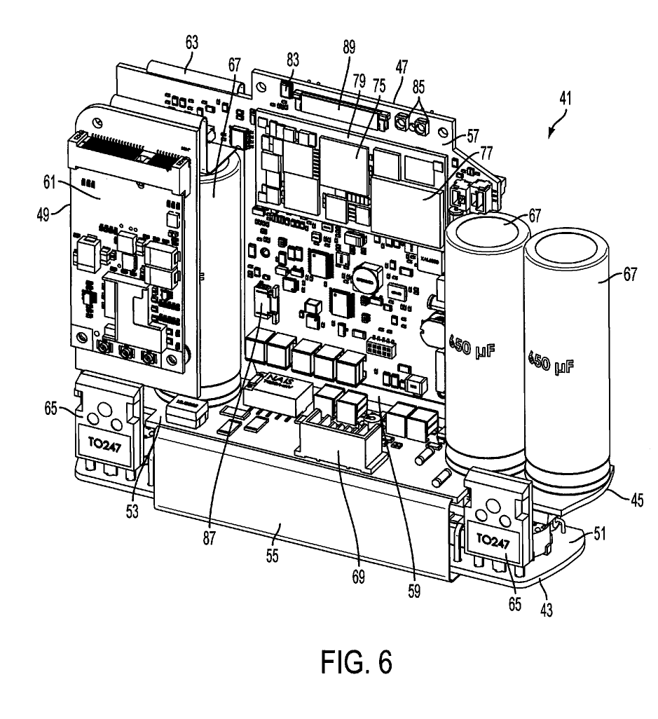

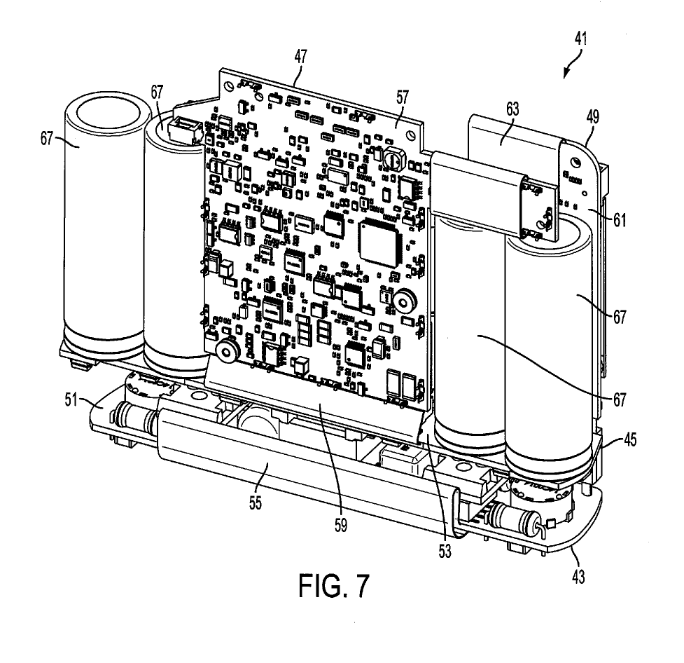

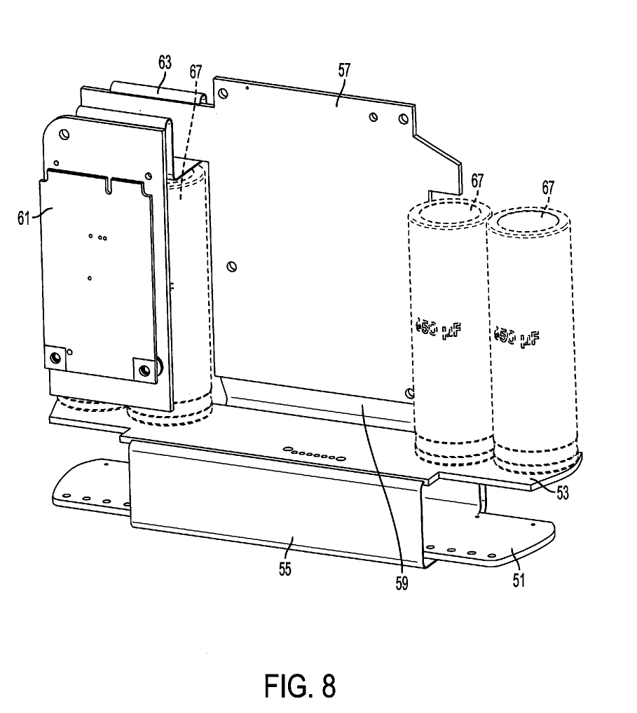

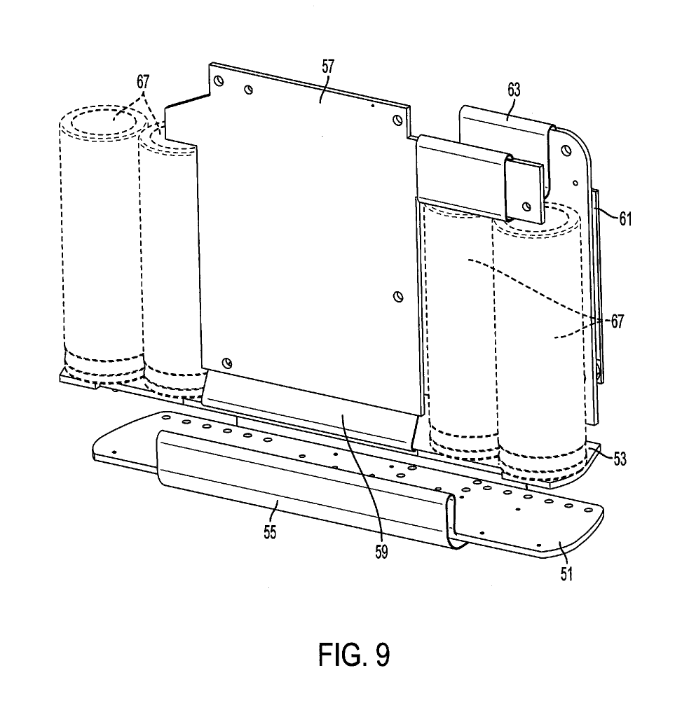

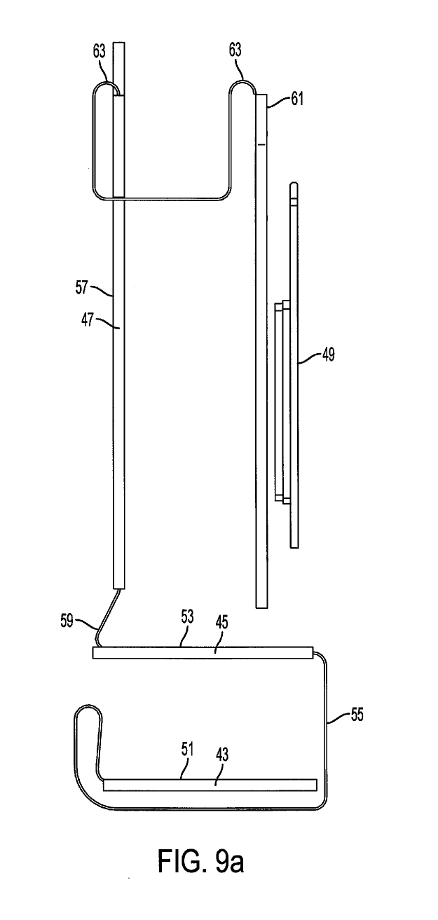

The first flexible member 55 is folded such that the first portion 51 of the distributed printed circuit board 41 is positioned substantially parallel to the second portion 53 of the distributed printed circuit board 41. The first flexible member 55 has a sufficient length to prevent the first portion 51 from colliding with the second portion 53 when the distributed circuit board 41 is folded into the folded configuration (see FIGS. 6 and 7). Accordingly, the first flexible member 55 is folded such that it has a substantially C-shaped cross-section. With reference to FIGS. 8 and 9, all of the components have been removed from the distributed circuit boards 41 such that the manner in which the first flexible member 55 is folded can be more easily observed.

The third portion 57 of the distributed printed circuit board 41 that encompasses the controller module 47 generally has a length in the range of about 3.5 to 4.5 inches and a width in the range of about 2.5 to 3.5 inches. This configuration of the third portion 57 allows it to be fit securely within a central portion of the external housing 3 substantially parallel to the front cover 7 and the rear cover 9 and substantially perpendicular to the first portion 51 and the second portion 53. The second flexible member 59 extending between the second portion 53 and the third portion 57 of the distributed printed circuit board 41 is folded such that the third portion 57 is positioned substantially perpendicular to the first portion 51 and the second portion 53 of the distributed printed circuit board 41. The second flexible member 59 is folded such that it has a substantially L-shaped cross-section. With reference to FIGS. 8 and 9, all of the components have been removed from the distributed circuit boards such that the manner in which the second flexible member 59 is folded can be more easily observed.

The controller module 47 may comprise a microprocessor and memory device 75 and an SD card holder 77 mounted on a separate printed circuit board 79 that is operatively connected to the third portion 57 of the distributed printed circuit board 41. The memory device is desirably flash memory. These elements can be operatively connected to the separate printed circuit board 79 by ball grid arrays (BGAs) that are located so as to be minimally affected by mechanical stress that can be transmitted through the defibrillator 1, for example, such as upon impact of a portion of the housing of the defibrillator 1 with a hard surface. The BGAs can be located upon a separate printed circuit board 79 and/or upon a portion of the distributed printed circuit board 41 that is not susceptible to excess flexing, for example, on the third portion 57 of the distributed printed circuit board 41. If the BGAs that control the memory were placed indiscriminately on the distributed printed circuit board 41, they would be more susceptible to flexing, ultimately breaking the brittle solder balls that make up the base of the component. By moving the BGAs to the separate printed circuit board 79, or by selecting a portion of the distributed printed circuit board 41 that is not susceptible to excess flexing, for example, on the third portion 57 of the distributed printed circuit board 41, an extra layer of protection for the BGAs is provided since they are isolated from the bending of the distributed printed circuit board 41 during impacts or other mechanical loads, making the design more rugged, and increasing longevity.

The BGAs can be secured to the separate printed circuit board 79 or the third portion 57 of the distributed printed circuit board 41 by a suitable adhesive, for example, by being under-filled with an epoxy material, such as Loctite 3536 epoxy, available from Henkel AG & Co. KGaA of Düsseldorf, Germany. This process allows for the epoxy material to flow under the BGAs and around the solder balls that make the electro-mechanical connections to the separate printed circuit board 79 to form a rigid and secure support for the BGAs. Once under-filled, the BGAs are subjected to stress shielding, which further protects them from flexing.

Finally, finite element analysis (FEA) was utilized to estimate the flexure of the boards during drop simulations. To set up the analysis, a fixed boundary was established on the side of the external housing 3 impacting the ground, and then a 400G gravity load was applied to the system in the direction of a fall. This is a quasi-static estimation of a dynamic load, but is generally accurate for a 40 foot drop. Once the external housing 3 and distributed printed circuit board 41 are assembled, and the simulation run, the results of the analysis illustrated the area on third portion 57 of the distributed printed circuit board 41 where the BGAs of the separate printed circuit board 79 should be mounted (i.e., away from major flexure points in the distributed printed circuit board 41, which were centered around the screw holes). By mounting the separate printed circuit board 79 in this area, the BGAs thereof are prevented from failing due to flexure, thereby making the defibrillator 1 resistant to drop failures. The above-described measures allow the defibrillator 1 to be highly durable and resistant to breaking.

Furthermore, the separate printed circuit board 79 may be accessed and replaced, if needed, through an access opening 81 provided in a rear portion of the battery slot 15 (see FIG. 3). This also allows a user to access an SD card from the SD card holder 77.

The microprocessor 75 is configured to receive digital or analog ECG information either directly or indirectly from the ECG electrodes (not shown) of the therapeutic device (not shown), detect abnormal heart rhythms based on the information received from the ECG electrodes, charge the capacitors 67 of the energy storage module 45, and control the energy discharge module 43 to administer a therapeutic shock to the patient, unless a user intervenes within a predetermined period of time via the response buttons 19. In at least one example, the predetermined period of time in which a user may intervene does not end until actual delivery of the therapeutic shock. An example of the methods used to detect abnormal heart rhythms can be found in U.S. Pat. No. 5,944,669, which is assigned to the assignee of the present application and which is hereby incorporated by reference in its entirety. Additionally, an example of the general features of a defibrillator can be found in U.S. Pat. No. 6,280,461, which is assigned to the assignee of the present application and which is also hereby incorporated by reference in its entirety.

The microprocessor 75 is also configured to perform several non-critical functions. These non-critical functions may leverage the robust computing platform provided by the microprocessor 75 without disrupting the critical functions of the defibrillator 1. Some examples of these non-critical functions include notifying emergency personnel of the location of a patient who just received a therapeutic shock via the communication module 49, providing users of the device with the historical physiological data of the wearer of the device via the display screen 25, and/or notifying the manufacturer of the defibrillator 1 of potential performance issues within the defibrillator 1 that may require repair to or replacement of the defibrillator 1 via the communication module 49. Moreover, these non-critical functions can include maintaining a history of data and events by storing this information in the memory device, communicating with the user via the display screen 25, and/or reporting data and events via the communication module 49. In addition, other non-critical functions may perform additional operations on the history of critical data. For instance, in one example, a non-critical function analyzes the history of critical data to predict worsening heart failure or an increased risk of sudden cardiac death.

The memory device of the defibrillator 1 is sized to store months or years of sensor information, such as ECG data, that is gathered over several monitoring and treatment periods. These monitoring and treatment periods may include continuous monitoring periods of approximately 23 hours (and substantially continuous monitoring periods of approximately 1-2 months) during which several treatments may be delivered to the patient. In some of these examples, the microprocessor 75 is configured to analyze the stored sensor information and to determine adjustments to the treatment method, or alternative treatment methods, of benefit to the patient. For instance, in one example, the microprocessor 75 is configured to analyze ECG data collected substantially contemporaneously with each instance of patient initiated delay, or cancellation, of treatment. In this example, the microprocessor 75 is configured to analyze the stored months of ECG data to recognize individualized, idiosyncratic rhythms that, while not normal, do not indicate a need for treatment. In some examples, the microprocessor 75 may automatically adjust the treatment method of the defibrillator 1 to better suit the patient by not initiating treatment in response to the recognized, idiosyncratic rhythm. Such an adjustment may be performed in conjunction with review by appropriate medical personnel.

Referring now to FIG. 6, the third portion 57 of the distributed printed circuit board 41 may further comprise a microphone 83 mounted thereon. A silicon gasket (not shown) may be provided to direct the audio through the microphone port 23 provided on the exterior of the defibrillator 1 to the microphone 83. In addition, a pair of LEDs 85 are mounted on the third portion 57 of the distributed printed circuit board 41. Light pipes can redirect the light from the LEDs 85 to a pair of icons (not shown) provided on the top cover 11 of the defibrillator 1.

A flex connector 87 for the touch screen of the display screen 25 and a flex connector 89 for the LCD of the display screen 25 can be mounted on the third portion 57 of the distributed printed circuit board 41. These connectors 87, 89 allow the display screen 25 to be operatively coupled to the third portion 57 of the distributed printed circuit board 41. Alternatively, one or more of the flex connectors 87, 89 can include a flexible portion of the distributed printed circuit board 41.

The fourth portion 61 of the distributed printed circuit board 41 that encompasses the communication module 49 may have a width that is greater than its length. Generally, it has a length in the range of about 0.5 to 1.5 inches and a width in the range of about 2.5 to 3.5 inches. This configuration of the fourth portion 61 allows it to be fit securely within the external housing 3 substantially parallel to the front cover 7 and the rear cover 9 and substantially perpendicular to the first portion 51 and the second portion 53. The third flexible member 63, extending between the third portion 57 and the fourth portion 61 of the distributed printed circuit board 41, is folded such that the fourth portion 61 is positioned substantially perpendicular to the first portion 51 and the second portion 53 of the distributed printed circuit board 41 and substantially parallel to the third portion 57 of the distributed printed circuit board 41. The third flexible member 63 is folded such that it has a substantially S-shaped cross-section, as shown in FIG. 9a. Alternatively to using the third flexible member 63 to connect the fourth portion 61 to the third portion 57, other connection methods could be utilized. For instance a perpendicular connector could be used to connect the fourth portion 61 to a top portion of the third portion 57 such that the fourth portion 61 is provided above the battery 13 and parallel to the second portion 53 and the first portion 51.

The communication module 49 provided on the fourth portion 61 of the distributed printed circuit board 41 provides various devices for communicating information to and from the defibrillator 1. For instance, the communication module 49 may include a GPS transceiver, a Bluetooth™ transceiver, a Wi-Fi transceiver, and/or a cellular transceiver. The communication module 49 is controlled by the controller module 47 to communicate information regarding the defibrillator 1 as discussed hereinabove.

A cellular antenna (not shown) for the cellular transceiver can be positioned within the external housing 3 of the defibrillator 1. The cellular antenna is optimized to have peak efficiency at the cell frequencies of several regions including, but not limited to, the United States, Japan, and Europe. The cellular antenna is located under the dragon trail lens of the display screen 25 and far enough away from the distributed printed circuit board 41 so that it can communicate efficiently. As shown in FIG. 6, in some embodiments, a metallic portion of the third portion 57 of the distributed printed circuit board 41 functions as part of the cellular antenna. Alternatively, a metallic portion of the display screen 25 of the defibrillator 1 may function as part of a cellular antenna for the cellular transceiver.

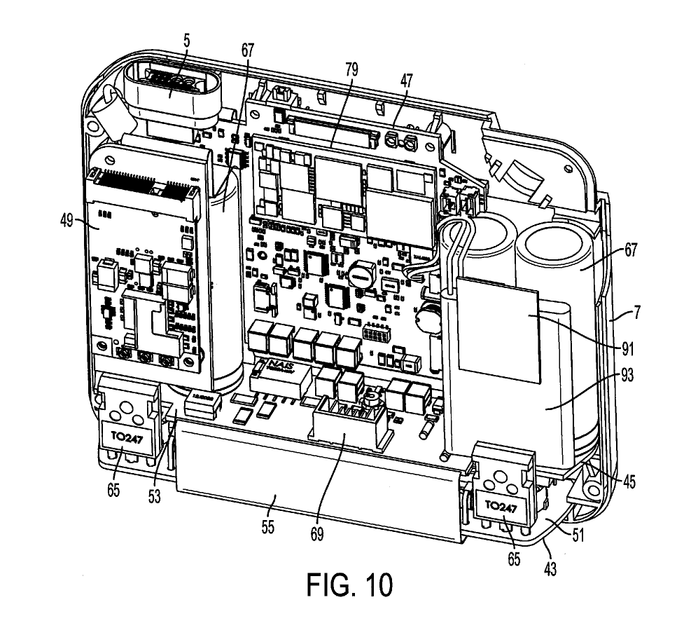

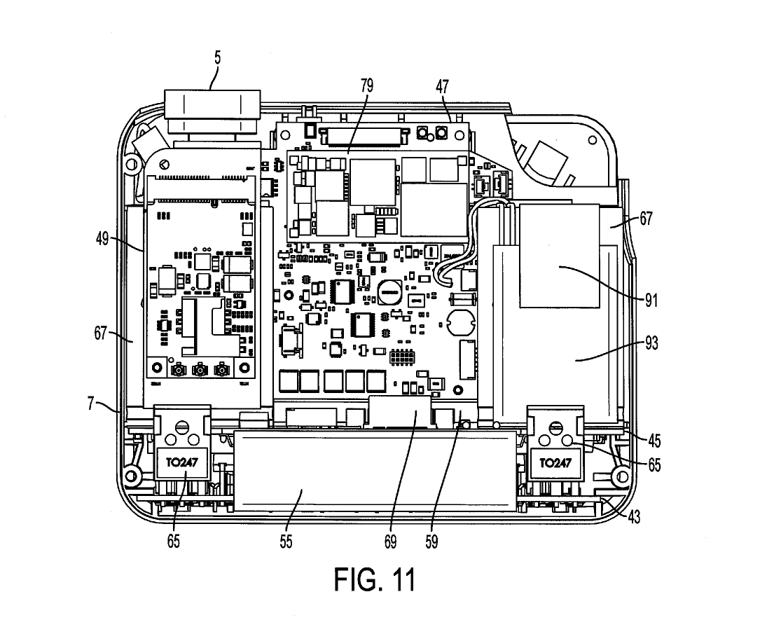

Similarly, an RFID antenna 91 (see FIGS. 10 and 11) may be positioned within the external housing 3 of the defibrillator 1 away from the four portions of the distributed printed circuit board 41 in order to communicate efficiently. The RFID antenna 91 is used to quickly communicate the identification of the defibrillator 1 to service personnel. In order to accommodate the RFID antenna 91, a backup battery 93 was positioned in the location shown in FIGS. 10 and 11.

With reference to FIGS. 10 and 11 and with continuing reference to FIGS. 1-9, the defibrillator 1 may be manufactured as follows. First, the distributed printed circuit board 41 is provided in the unfolded configuration as shown in FIG. 4. Thereafter, the first flexible member 55 is folded such that the first portion 51 of the distributed printed circuit board 41 is positioned substantially parallel to the second portion 53 of the distributed printed circuit board 41. Next, the second flexible member 59 is folded such that the third portion 57 of the distributed printed circuit board 41 is positioned substantially perpendicular to the first and second portions 51, 53 of the distributed printed circuit board 41. Then, the third flexible member 63 is folded such that the fourth portion 61 of the distributed printed circuit board 41 is positioned substantially parallel to the third portion 57 of the distributed printed circuit board 41 and substantially perpendicular to the first and second portions 51, 53 of the distributed printed circuit board 41, thereby providing a folded distributed circuit board as shown in FIGS. 6 and 7. When folded in this manner, the high voltage energy storage module 45 and discharge module 43 are isolated from the low voltage controller module 47 and communication module 49. In addition, by positioning the communications module 49 in this manner, interference between the components of the communications module 49 and other components of the device can be substantially avoided and eliminated.

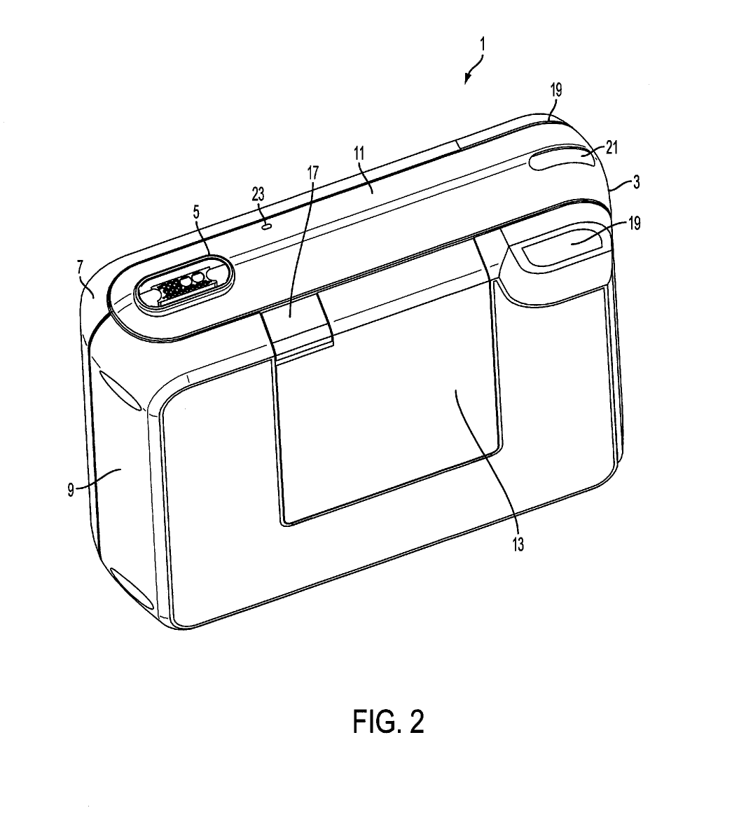

Next, the front cover 7, rear cover 9, and top cover 11 are provided. The folded distributed printed circuit board 41 is positioned within the front cover 7 and secured thereto via an appropriate fastening device, such as screws. Finally, the top cover 11 is positioned in the appropriate location and the rear cover 9 is secured to the front cover 7 and top cover 11 using any appropriate fastening device. This produces a defibrillator 1 as shown in FIGS. 1 and 2.

Accordingly, a defibrillator 1 is provided that has a small footprint, is very durable, and can be used in a variety of patient care scenarios where a conventional implantable cardioverter-defibrillator cannot. Examples of these scenarios include treatment when the patient is awaiting a pending transplant or where the patient has a systemic infection (e.g., influenza or osteomyelitis), myocarditis, intra-ventricular thrombus, cancer, or a life-limiting serious illness such that an implantable device is not medically prudent.

Although a defibrillator 1 has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements. For example, it is to be understood that this disclosure contemplates that, to the extent possible, one or more features of any embodiment can be combined with one or more features of any other embodiment.

The invention claimed is:

1. A defibrillator comprising:
a housing; and
a printed circuit board comprising a plurality of portions operatively connected by flexible members, the plurality of portions further comprising:
a first portion of the printed circuit board upon which may be disposed a discharge module for selectively delivering an energy pulse to a patient;
a second portion of the printed circuit board upon which may be disposed an energy storage module; and
a third portion of the printed circuit board upon which may be disposed a controller module for controlling delivery of the energy pulse to the patient,
wherein the flexible members are folded such that at least one of the plurality of portions of the printed circuit board is positioned substantially perpendicular to at least one of the others of the plurality of portions of the printed circuit board, thereby allowing the discharge module, energy storage module, and controller module to be positioned within the housing.

2. The defibrillator of claim 1, wherein the flexible members comprise at least a first flexible member connecting the first portion of the printed circuit board to the second portion of the printed circuit board.

3. The defibrillator of claim 2, wherein the first flexible member is folded such that the first portion of the printed circuit board is positioned substantially parallel to the second portion of the printed circuit board.

4. The defibrillator of claim 1, wherein the flexible members comprise at least a second flexible member connecting the second portion of the printed circuit board to the third portion of the printed circuit board.

5. The defibrillator of claim 4, wherein the second flexible member is folded such that the third portion of the printed circuit board is positioned substantially perpendicular to the first and second portions of the printed circuit board.

6. The defibrillator of claim 1, wherein the plurality of portions further comprise a fourth portion of the printed circuit board upon which may be disposed a communication module.

7. The defibrillator of claim 6, wherein the flexible members comprise at least a third flexible member connecting the fourth portion of the printed circuit board to the third portion of the printed circuit board.

8. The defibrillator of claim 7, wherein the third flexible member is folded such that the fourth portion of the printed circuit board is positioned substantially parallel to the third portion of the printed circuit board and substantially perpendicular to the first and second portions of the printed circuit board, thereby allowing the communication module to be positioned within the housing.

9. The defibrillator of claim 6, wherein the communication module includes at least one of a GPS transceiver, a Bluetooth transceiver, a Wi-Fi transceiver, and a cellular transceiver.

10. The defibrillator of claim 1, wherein the controller module comprises a memory and a microprocessor.

11. The defibrillator of claim 10, wherein the memory and microprocessor are operatively connected to a separate printed circuit board that is operatively connected to the third portion of the printed circuit board.

12. The defibrillator of claim 11, wherein the memory and microprocessor are operatively connected to the separate printed circuit board by ball grid arrays that are under-filled with an epoxy material.

13. The defibrillator according to claim 10, wherein the memory and microprocessor are operatively connected to the third portion of the printed circuit board by ball grid arrays that are under-filled with an epoxy material.

14. The defibrillator according to claim 1, wherein at least one of a metallic portion of the third portion of the distributed printed circuit board and a metallic portion of a display of the defibrillator functions as part of a cellular antenna for a cellular transceiver.

15. The defibrillator of claim 1, further comprising a display positioned on an exterior surface of the housing for providing a user interface.

16. The defibrillator of claim 1, further comprising a speaker mounted within an air-filled reverberator positioned at a corner of the housing.

17. The defibrillator of claim 1, wherein the housing includes a port for connecting the defibrillator to a treatment device.

18. The defibrillator of claim 1, further comprising a power supply configured to be positioned within a dock provided on the housing and operatively connected to a member provided on the energy storage module.

19. A defibrillator comprising:
a housing;
at least one high-voltage module; and
at least one low-voltage module operatively connected to the at least one high-voltage module by at least one flexible member,
wherein the at least one flexible member is folded such that the at least one high-voltage module and the at least one low-voltage module are positioned within the housing and such that spacing between the at least one high-voltage module and the at least one low-voltage module provides at least one of isolation of high-voltage from low voltage and minimizes interference between the at least one high-voltage module and the at least one low-voltage module.

20. The defibrillator of claim 19, wherein the at least one high-voltage module includes at least one of a discharge module for selectively delivering an energy pulse to a patient and an energy storage module for storing an energy pulse to be delivered to the patient.

21. The defibrillator of claim 19, wherein the at least one low-voltage module includes at least one of a controller module for controlling delivery of an energy pulse to a patient and a communication module for allowing the defibrillator to communicate with an external device.

22. The defibrillator of claim 21, wherein the controller module is positioned within the housing at a location such that stresses caused by external forces on the housing are reduced.

23. The defibrillator of claim 21, wherein the controller module comprises a memory and a microprocessor.

24. The defibrillator of claim 23, wherein the memory and microprocessor are operatively connected to a separate printed circuit board that is operatively connected to the third portion of the printed circuit board.

25. The defibrillator of claim 23, wherein the memory and microprocessor are operatively connected to the separate printed circuit board by ball grid arrays that are under-filled with an epoxy material.

26. A method of manufacturing a defibrillator comprising:
providing a distributed circuit board comprising:
a discharge module disposed on a first portion of a printed circuit board for selectively delivering an energy pulse to a patient;
an energy storage module disposed on a second portion of the printed circuit board that is operatively connected to the discharge module by a first flexible member; and
a controller module for controlling delivery of the energy pulse to the patient disposed on a third portion of the printed circuit board that is operatively connected to the energy storage module by a second flexible member;
folding the first flexible member such that the first portion of the printed circuit board is positioned substantially parallel to the second portion of the printed circuit board;
folding the second flexible member such that the third portion of the printed circuit board is positioned substantially perpendicular to the first and second portions of the printed circuit board, thereby providing a folded distributed circuit board;
providing a housing having an upper housing portion and a lower housing portion;
positioning the folded printed circuit board within one of the upper housing portion and the lower housing portion; and
securing the upper housing portion to the lower housing portion.

27. The method of claim 26, wherein the distributed circuit board further comprises a communication module disposed on a fourth portion of the printed circuit board and operatively connected to the controller module by a third flexible member, and
the method further comprises folding the third flexible member such that the fourth portion of the printed circuit board is positioned substantially parallel to the third portion of the printed circuit board and substantially perpendicular to the first and second portions of the printed circuit board.

28. A defibrillator comprising:
a housing;
a plurality of operatively connected modules; and
a printed circuit board comprising a plurality of portions operatively connected by flexible members, the plurality of portions further comprising:
a first portion of the printed circuit board upon which may be disposed at least one of a discharge module for selectively delivering an energy pulse to a patient-and an energy storage module; and
a second portion of a printed circuit board upon which may be disposed at least one of a controller module for controlling delivery of the energy pulse to the patient and a communication module for allowing the defibrillator to communicate with an external device;
wherein at least one of the flexible members are folded such that the first portion is positioned substantially perpendicular to the second portion, thereby allowing the plurality of operatively connected modules to be positioned within the housing.

29. A defibrillator comprising:
a housing;
a plurality of operatively connected modules comprising
a discharge module for selectively delivering an energy pulse to a patient,
an energy storage module, and
a controller module for controlling delivery of the energy pulse; and
a printed circuit board comprising a plurality of portions operatively connected by flexible members, the plurality of portions further comprising:
a first portion of a printed circuit board upon which may be disposed at least one of the plurality of modules; and
a second portion of a printed circuit board upon which may be disposed at least one of a remainder of the plurality of modules;
wherein at least one of the flexible members are folded such that the first portion is positioned substantially perpendicular to the second portion, thereby allowing the plurality of operatively connected modules to be positioned within the housing.

30. The defibrillator of claim 29, wherein the at least one of the plurality of modules disposed on the first portion comprises at least one of the discharge module and the energy storage module.

31. The defibrillator of claim 30, wherein the at least one of the remainder of the plurality of modules disposed on the second portion comprises the controller module.

32. The defibrillator of claim 30, wherein the at least one of the plurality of modules disposed on the second portion comprises at least one of the controller module and a communication module for allowing the defibrillator to communicate with an external device.

33. The defibrillator of claim 29, wherein the at least one of the remainder of the plurality of modules disposed on the second portion comprises the controller module.

34. The defibrillator of claim 29, wherein the at least one of the plurality of modules disposed on the second portion comprises at least one of the controller module and a communication module for allowing the defibrillator to communicate with an external device.
